(19) United States
(12) Patent Application Publication
(10) Pub. No.: US 2026/0069611 A1
(43) Pub. Date: Mar. 12, 2026

(54) STABLE CANNABIDIOL ORAL FORMULATIONS

(71) Applicant: A2W Pharma Limited

(72) Inventors: John Paul MIZZI, Sannat (MT); Domenico GURRADO, Marsaxlokk (MT)

(21) Appl. No.: 18/827,370

(22) Filed: Sep. 6, 2024

(51) Int. Cl. A61K 31/00; A61K 9/00; A61K 36/185; A61K 47/10; A61K 47/22; A61K 47/24; A61K 47/44

(52) U.S. Cl. CPC A61K 31/658; A61K 9/0053; A61K 36/3482; A61K 47/10; A61K 47/22; A61K 47/24; A61K 47/44

(57) ABSTRACT

A stable oral composition comprising of: a) Cannabidiol; b) DL-α tocopherol; c) a cosolvent selected from Diethylene glycol mono ethyl ether (Transcutol-HPR) or the surfactant agent Lecithin; d) a synthetic sweetener; e) a flavoring agent and; f) Corn oil as a solvent. A stable oral composition comprising of (i) cannabidiol (CBD) at a concentration of from about 95 mg/ml to about 105 mg/ml, (ii) Diethylene glycol mono ethyl ether at a concentration from about 4.5 mg/ml to about 5.5 mg/ml or Lecithin at a concentration from about 0.45 mg/ml to about 0.55 mg/ml, (iii) DL-α tocopherol at a concentration from about 0.54 mg/ml to about 6.6 mg/ml, (iv) synthetic sweetener at a concentration from about 0.018 mg/ml to about 0.022 mg/ml, (v) Flavoring agent at a concentration from about 0.9 mg/ml to about 1.1 mg/ml, and (vi) corn oil q.s. to about 1.0 ml.

### FIELD

The present invention describes stable oral formulations of cannabidiol (CBD) for therapeutic use. The invention illustrates development of oral cannabidiol formulations and manufacturing process having improved stability and palatability.

### BACKGROUND

Cannabidiol (CBD) is a phytocannabinoid discovered in 1940. It is one of 113 identified cannabinoids in cannabis plants, along with tetrahydrocannabinol (THC), and accounts for up to 40% of the plant's extract.

Cannabidiol is reported for use in treatment of anxiety, addiction, psychosis, movement disorders, pain and the like. Some of them as described in the literature are as mentioned below. Cannabidiol may be suitable for the symptoms or treatment of diseases or disorders, such as, Lennox Gastaut Syndrome, Dravet Syndrome, myoclonic seizures, schizophrenia, juvenile spasms, refractory infantile spasms, tubular sclerosis complex, brain tumours, neuropathic pain, cannabis use disorder, post-traumatic stress disorder, anxiety, early psychosis, Alzheimer's Disease autism, and withdrawal from opioids, cocaine, heroin, amphetamines, and nicotine and the like.

Cannabinoids are also synthesized in laboratory thereby eliminating the need to grow cannabis for extraction of the compounds.

One cannabinoid, cannabidiol, (−)-trans-2-p-mentha-1,8-dien-3-yl-5-pentylresorcinol, is non-psychoactive and has shown promise in treating numerous diseases and disorders. Synthetic cannabidiol has the same structure as naturally occurring cannabidiol.

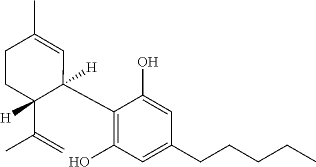 [SMILES: C=C(C)[C@@H]1CCC(C)=C[C@H]1c1c(O)cc(CCCCC)cc1O]

Cannabidiol is water-insoluble and highly lipophilic in nature. Literature reports the administration of cannabidiol using different dosage forms viz. inhalation, capsules, dried cannabis, liquid oral solution, oil, etc. However, being water-insoluble, it's always a task to formulate the same at higher doses (as required) in oral dosage forms.

In September 2018, the GW Pharmaceuticals product-Epidiolex® was placed in Schedule V of the Controlled Substances Act by the Drug Enforcement Administration (DEA), following its approval by the FDA for rare types of childhood epilepsy as mentioned above. It was then removed from the Controlled Substances Act by the DEA in April 2020. USFDA approved cannabidiol (Epidiolex®) as oral solution in 2018 for the treatment of seizures associated with Dravet syndrome, Lennox-Gastaut syndrome, or tuberous sclerosis complex in people. Epidiolex® is available for prescription use in all states.

EPIDIOLEX® (cannabidiol) oral solution is a clear, colourless to yellow liquid containing 100 mg/mL CBD dissolved in the following excipients: sesame oil and anhydrous ethanol (79 mg/mL) with added sweetener (sucralose [0.5 mg/mL]) and strawberry flavouring (0.2 mg/mL).

Commercially available cannabidiol is usually contaminated with delta 9-tetrahydrocannabinol. Further, delta-9-tetrahydrocannabinol is a Psychoactive and patients receiving cannabidiol wish to avoid this undesirable side effect of the delta-9-tetrahydrocannabinol contaminant. Therefore, there is a need for a substantially pure cannabidiol either synthetically synthesized or obtained from plant source which does not contain delta-9-tetrahydrocannabinol.

However, many attempts in the recent literature have been reported for developing cannabinoid stable formulations. Few of them have been listed below:

### PRIOR ART

As mentioned above, the literature reports quite a few cannabidiol (CBD) formulations with varying dosages depending upon its intended use (either in treatment of seizures or in neuropathic pain or in removal of addiction, etc).

For example,

U.S. Pat. No. 6,946,150B2 describes oral spray formulations with Cannabinoid comprising a solvent/co-solvent and flavoring agent. However, these formulations were lacking specific sweeteners/taste masking agents and face challenges regarding CBD solubility due to its high lipophilicity, as reported in the patent.

U.S. Pat. No. 6,383,513 explore intranasal microsphere delivery system comprising cannabinoid which is encapsulated/dispersed on the surface of a microsphere carrier matrix. It also describes compositions comprising cannabinoids, wherein a cannabinoid (e.g., THC) was solubilized in sesame oil, castor oil, Vitamin E, etc; however, the concentration that was solubilized in sesame oil was about 35 mg/ml only.

Other patents, like U.S. Pat. Nos. 6,383,513 and 11,331,279B2, explore microsphere delivery systems and stable oral compositions of cannabinoids respectively and the use of sesame oil and lipids to solubilize cannabinoid (e.g. THC).

Despite these efforts, achieving stable and palatable oral formulations remains a challenge, particularly without the inclusion of alcohol, which may be unsuitable for pediatric patients, as described in U.S. Pat. Nos. 11,224,660 and 11,331,279.

In 2018, GW was successful in solubilizing higher doses of CBD using sesame oil and launched commercial oral formulation of CBD viz Epidiolex®.

However, these formulations still rely on ethanol as a co-solvent, potentially compromising their suitability for paediatric use and introducing impurities from CBD synthesis.

Thus, there remains a need for stable, palatable, and preferably alcohol-free oral compositions of CBD. The instant invention aims to address this need by developing a formulation that overcomes the challenges presented by prior art. The proposed composition is alcohol-free and formulated with critical excipient ratios to stabilize CBD, ensure consistent dosage, and enhance palatability, particularly for pediatric patients requiring treatment for seizures.

Therefore, the present invention seeks to develop a stable pharmaceutical composition of CBD using a straightforward and cost-effective manufacturing process, offering a novel solution to the challenges faced by existing CBD formulations.

### OBJECT AND SUMMARY OF THE INVENTION

It is an object of the present invention to formulate stable pharmaceutical composition comprising cannabidiol with enhanced stability.

It is an object of the present invention to develop a pharmaceutical composition with enhanced stability and palatability, utilising specific excipients, including one or more stabilizers, and employing a simple and cost-effective manufacturing process.

It is an object of the present invention to develop a pharmaceutical composition with enhanced stability utilizing one or more stabilizers.

Yet another object of the present invention is to design a pharmaceutical composition comprising an effective amount of cannabidiol in combination with a pharmaceutically acceptable carrier(s). The composition can be in the form of an oral solution.

In another aspect, the present invention is directed to stable pharmaceutical formulations for oral administration comprising of cannabinoid, solvent, co-solvent along with sweetener, flavouring agent and an antioxidant.

In another aspect, the present invention is directed to stable pharmaceutical formulations for oral administration comprising cannabidiol and oily vehicle or lipids wherein the formulation is free of alcohol.

These objectives drive the invention, which aims to provide pharmaceutical compositions of Cannabidiol that overcome limitations related to stability, and palatability. By achieving these objectives, the present invention offers novel and improved formulations of cannabidiol for enhanced therapeutic outcomes.

The following embodiments further describe the objects of the present invention in accordance with the best mode of practise. However, the disclosed invention is not restricted to the embodiments hereinafter described.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1A is a diagram showing the CBDQ trend and prediction at 40° C./75% RH of Stable Oral Composition.

FIG. 1B is a diagram showing the CBDQ trend and prediction at 40° C./75% RH of Epidiolex®.

FIG. 2A is a diagram showing CBD purity trend and prediction at 40° C./75% RH of Stable Oral Composition.

FIG. 2B is a diagram showing the CBD purity trend and prediction at 40° C./75% RH of Epidiolex®.

### DETAILED DESCRIPTION

The present invention can be more readily understood by reading the following detailed description of the invention and studying the included examples.

“Pharmaceutical composition” and composition,” as used herein, are equivalent terms referring to a composition of active ingredients for pharmaceutical use.

“Stable cannabidiol solution”, as used herein means stability of solution against impurities which arise from cannabidiol.

As used herein, “synthetic” refers to the chemically synthesized cannabidiol and it does not refer to cannabidiol that is extracted from plant source.

As used herein, “substantially pure” refers to a preparation having chromatographical purity of cannabidiol of greater than 99.0%.

As used herein, all numerical values relating to amounts, weights, and the like are defined as “about”. Each value is plus or minus 10%. For example, the phrase “about 10% w/w” is to be understood as “9% to 11% w/w.” Therefore, amounts within 10% of the claimed value are encompassed by the scope of the claims. As used herein, “% w/w” refers to the percent weight of the total composition. As used herein, the term “effective amount” refers to the amount necessary to treat a patient in need thereof.

The present invention shows various advantages over conventional formulations, like:
1. The physical stability enhancement obtained by this method impart several desirable characteristics to the products, such as longer-term physical stability to the product and chemical stability to cannabidiol.
2. The simplicity and cost-effectiveness of the technology
3. Improved palatability of drug.

In an embodiment the present invention a stable pharmaceutical formulation for oral administration comprises of cannabinoid, a solvent, sweetener, anti-oxidant, at least one co-solvent and a flavouring agent wherein the formulation does not contain alcohol.

In one embodiment of the present invention, a stable oral composition comprising:
a) Cannabidiol
b) DL-alpha tocopherol
c) Diethylene glycol mono ethyl ether (Transcutol-HP®) as co-solvent
d) a synthetic sweetener
e) a flavoring agent and
f) Corn oil as a solvent

In another embodiment the present invention a stable oral composition for administration comprises from about 95 mg/ml to about 105 mg/ml of a cannabinoid, from about 0.018 mg/ml to about 0.022 mg/ml of Sweetener, and from about 0.54 mg/ml to about 6.6 mg/ml of DL-alpha tocopherol, from about 0.9 mg/ml to about 1.1 mg/ml flavouring agent, and from about 4.5 mg/ml to 5.5 mg/ml co-solvent wherein the formulation does not contain alcohol.

In another embodiment of the present invention, a stable oral composition comprising:
a) 95 mg/ml to about 105 mg/ml Cannabidiol,
b) about 0.018 mg/ml to about 0.022 mg/ml of Neotame
c) about 0.54 mg/ml to about 6.6 mg/ml of D-alpha tocopherol
d) about 4.5 mg/ml to 5.5 mg/ml Diethylene glycol mono ethyl ether (Transcutol-HP®)
e) about 0.9 mg/ml to about 1.1 mg/ml Ethyl vanillin
f) Corn oil Q.S.

In another embodiment of the present invention, a stable oral composition comprising:
a) 95 mg/ml to about 105 mg/ml Cannabidiol,
b) about 0.018 mg/ml to about 0.022 mg/ml of Neotame
c) about 0.54 mg/ml to about 6.6 mg/ml of DL-alpha tocopherol
d) about 0.45 mg/ml to 0.55 mg/ml Lecithin
e) about 0.9 mg/ml to about 1.1 mg/ml of Ethyl vanillin
f) Corn oil Q.S.

In a preferred embodiment of the present invention, a stable oral composition comprising:
a) Cannabidiol about 100 mg/ml,
b) about 0.02 mg/ml of Neotame
c) about 6 mg/ml of D-alpha tocopherol
d) about 4.95 mg/ml Diethylene glycol monoethyl ether (Transcutol-HP®)
e) about 1 mg/ml Ethyl vanillin
f) Com oil Q.S.

In other preferred embodiment of the present invention, a stable oral composition consisting essentially of:
a) CBD at a concentration of between about 95 mg/ml and about 105 mg/ml, wherein the CBD has a purity of at least about 99.0% of purity and 98.0-105.0% w/w of assay;
b) Diethylene glycol mono ethyl ether (Transcutol-HP®) at a concentration of about 4.95 mg/ml;
c) Neotame at a concentration of about 0.02 mg/ml;
d) DL-alpha tocopherol at a concentration of about 6 mg/ml;
e) Ethyl Vanillin at a concentration of about 1 mg/ml; and
f) Corn oil, q.s. to about 1.0 ml.

In another preferred embodiment of the present invention, a stable oral composition consisting essentially of:
a) CBD at a concentration of between about 95 mg/ml and about 105 mg/ml, wherein the CBD has a purity of at least about 99.0% of purity and 98.0-105.0% w/w of assay;
b) Lecithin at a concentration of about 0.50 mg/ml;
c) Neotame at a concentration of about 0.02 mg/ml;
d) DL-alpha tocopherol at a concentration of about 6 mg/ml;
e) Ethyl Vanillin at a concentration of about 1 mg/ml; and
f) Corn oil, q.s. to about 1.0 ml.

In an embodiment of the present invention, the cannabidiol solution is devoid of alcohol.

In an embodiment of the present invention, the dose of cannabidiol in the pharmaceutical composition ranges from about 95 mg/ml to 105 mg/ml.

In yet another embodiment of present invention, formulations consist of cannabidiol that is substantially pure and is obtained through natural source and has a purity of greater than 99.0% of purity and 98.0-105.0% w/w of assay.

The impurities associated with cannabidiol primarily originate from the cannabidiol API itself, except for cannabidiol hydroquinone, which forms as a degradation product when cannabidiol is exposed to oxidative conditions.

In one embodiment of the invention, both cannabidiol hydroquinone and butyl cannabidiol impurities were detected at minimal levels.

In a further embodiment the CBD is obtained from a highly purified extract of cannabis which comprises at least 99.0% of purity and 98.0-105.0% w/w of assay.

In another embodiment of the present invention, the cannabidiol solution is prepared using the steps of:
1. Dissolving neotame in diethylene glycol monoethyl ether.
2. Dissolving ethyl vanillin and Alpha-tocopherol in corn oil.
3. Dissolving the Cannabidiol in corn oil under nitrogen at 25° C.
4. Adding the neotame solution to the mixture from Steps 2 and 3.
5. Checking the assay and adjust with additional corn oil or cannabidiol as needed.
6. Bottling the solution.

Once the composition is ready; the initial testing is done after filling the same in desired container/packaging. The same is then subjected to room temperature and accelerated stability studies and is monitored at regular intervals as in ICH guidelines. The Epidiolex® solution is also subjected to same stability studies for comparative analysis.

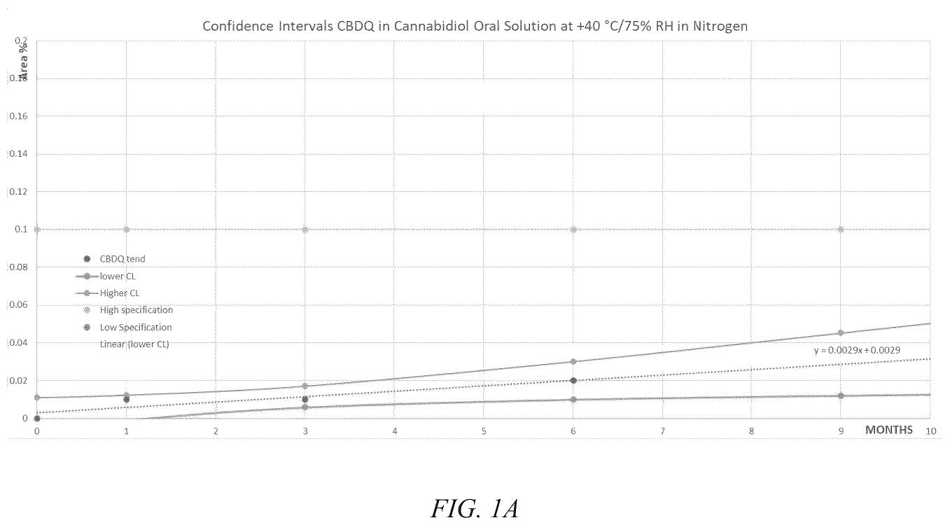

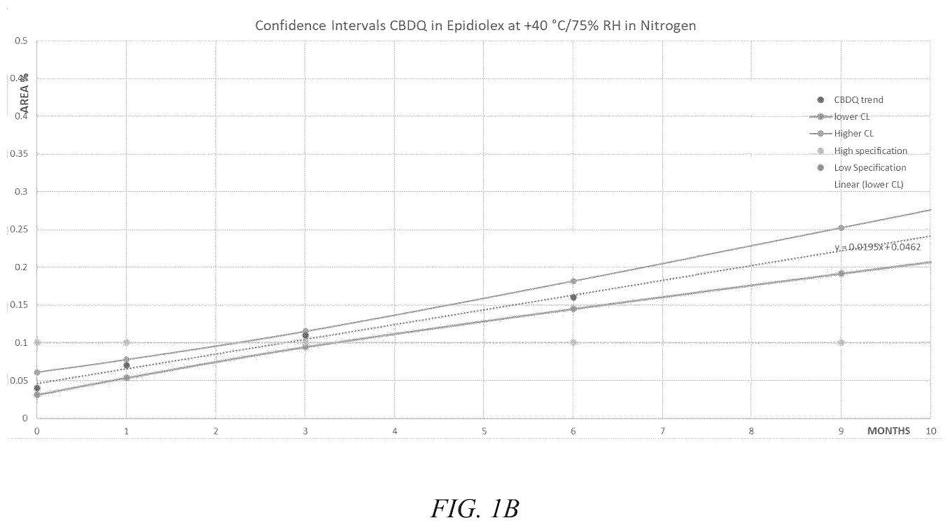

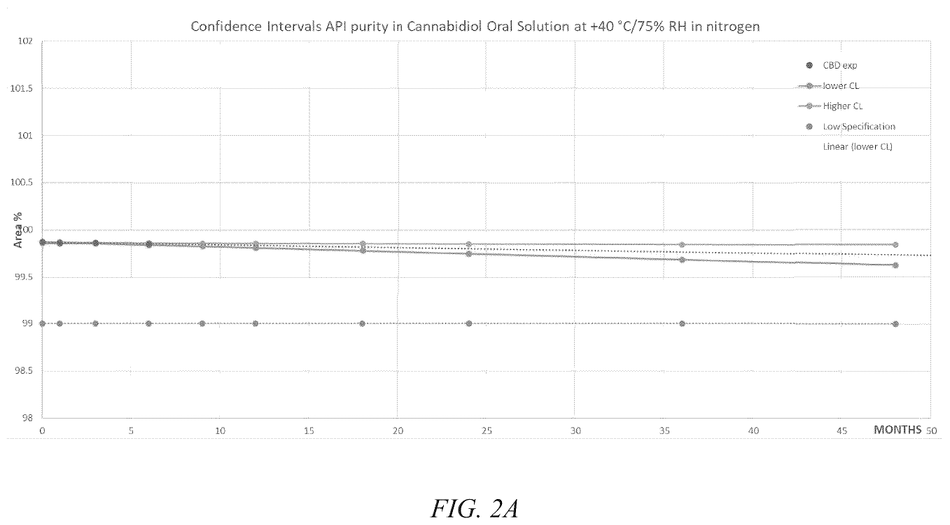

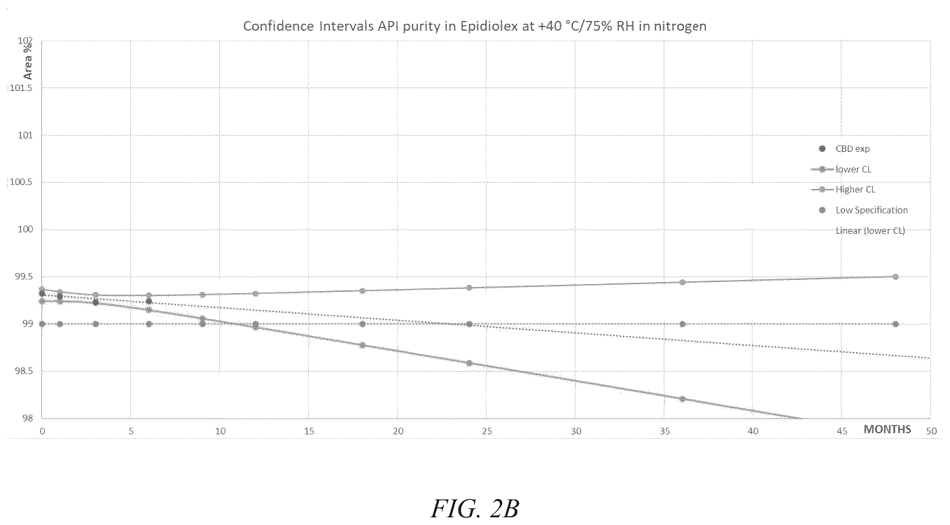

Thus, in the preferred embodiment of the present invention, the cannabidiol solution prepared using the above-mentioned process yields a stable formulation. The same is demonstrated by the stability study results as in Table-2A and 2B and comparative stability studies mentioned as in Table 3A, 3B and FIG. 1A, FIG. 1B, FIG. 2A and FIG. 2B shows that the present invention formulation imparts more stability and palatability to the formulation.

Hence, in a preferred embodiment of the present invention, the cannabidiol formulation is stable and palatable as compared to Epidiolex® oral solution.

In another embodiment of the present invention, the composition of present invention has no food effect.

In yet another embodiment of the present invention, the stable oral cannabidiol composition comprises of one or more pharmaceutically acceptable solvents, co-solvents, antioxidants, sweeteners and flavours or combination of these and the like.

In another embodiment of the present invention, the composition is administered to the patients suffering from seizures—either children or young adults. Cannabidiol solution is particularly effective in reducing atonic seizures in patients suffering with etiologies including Lennox-Gastaut Syndrome; Tuberous Sclerosis Complex; Dravet Syndrome; Doose Syndrome; Aicardi syndrome and the like and is also used in symptomatic relief of moderate to severe neuropathic pain or other painful conditions, like cancer.

The cannabidiol solution of the present invention is a liquid oral formulation wherein the drug is solubilized in oily vehicle/solvent.

In another embodiment of the present invention, the CBD solution imparts enhanced stability and helps to overcome the stability issue.

Cannabidiol (CBD) is highly lipophilic compound with poor oral bioavailability (as low as 6%), and has low water solubility (0.7 μg/mL) and extensive first-pass metabolism. Hence, various lipids and oils have been used for solubilizing the same till date.

As the prior arts reported, the medium and long chain triglycerides have also been used till date. However, medium-chain triglycerides can quickly absorb because it doesn't have to break it down via digestion before sending it off to the lymph system. Also, it absorbs easily through the skin.

Long-chain triglycerides require more digestion time. Short-chain triglycerides are often consumed by gut bacteria before they've had time to be absorbed. So MCTs are the most useful. However, comparatively edible oils like corn or maize oil or MCT (coconut oil) enable quick absorption due to molecular structure, aids in absorption, is almost flavorless, less expensive, etc.

Hence, in a preferred embodiment, the oil is selected from the group consisting of sesame oil, olive oil, corn oil, sunflower oil, safflower oil, flaxseed oil, almond oil, peanut oil, walnut oil, cashew oil, castor oil, coconut oil, palm oil, soybean oil, canola oil, vegetable oil, rice bran oil, and the combinations thereof. In a more preferred embodiment, the oil is corn oil or combinations thereof. In a most preferred embodiment, the oil used is corn oil.

The literature reports the use of co-solvent to enhance the solubility of cannabidiol. Epidiolex® also uses ethanol as co-solvent to solubilize CBD as well as sweeteners and flavouring agents. Other prior art reports the use of ethanol, propylene glycol, glycerine, etc as co-solvents. The CBD oral solution is used for paediatric patients and hence, is important to use ethanol either in very less quantity or eliminate the same.

Thus, in a preferred embodiment of the present invention, Diethylene glycol mono ethyl ether (Transcutol-HP®) and/or lecithin (soy) is used which aids in to increase the solubility and in turn bioavailability of cannabidiols.

In more preferred embodiment, diethylene glycol mono ethyl ether is used as co-solvent in the concentration from about 4.5 mg/ml to about 5.5 mg/ml.

In another preferred embodiment, lecithin is used as surfactant in the concentration of about 0.45 mg/ml to about 0.55 mg/ml.

In yet another embodiment of the present invention, one or more antioxidants are used to further enhance the stability of the composition.

Examples of suitable anti-oxidants include α-tocopherol (Vitamin E), D-α-tocopherol polyethylene glycol succinate (Vitamin E TPGS), ascorbic acid, sodium ascorbate, ascorbic palmitate or combinations thereof.

In a preferred embodiment, antioxidant used is DL-α-tocopherol (Vitamin E), D-α-tocopherol polyethylene glycol succinate (Vitamin E TPGS) or combinations thereof. In more preferred embodiment, DL-α-tocopherol is used as antioxidant in the concentration from about 0.54 mg/ml to about 6.6 mg/ml. Cannabidiol degrades to cannabidiol hydroxyquinone with exposure to light, oxygen and heat by oxidation and the anti-oxidant action of the DL-α-tocopherol reduces the rate of oxidation of cannabidiol to cannabidiol hydroxyquinone due to its anti-oxidant action. This helps to keep the assay of cannabidiol high and impurities of cannabidiol low, such as cannabidiol hydroxyquinone, throughout the shelf life of the product. The pharmaceutical composition of the present invention may also include sweeteners and favouring agents to enhance the palatability of cannabidiol in oils used within the composition. Examples of sweeteners include any natural or artificial sweetener, For example, sucrose, xylitol, sodium saccharin, neotame, cyclamate, aspartame, acesulfame, saccharin, erythritol and sucralose and combinations thereof. In certain embodiments, the sweetener is present in an amount from about 0.018 mg/ml to about 0.022 mg/ml. In more preferred embodiment, neotame is used as sweetener in the concentration from about 0.018 mg/ml to about 0.022 mg/ml.

Those or ordinary skill in the art will appreciate that the amount of sweetener may vary depending on the strength of the sweetener used and the levels approved by the regulatory authorities for use in pharmaceutical products.

Examples of flavouring agents include natural and artificial flavours, for example fruit flavours such as orange, grape, cherry, strawberry, lemon, lime, and the like and Limonene, methyl anthranilate, ethyl decadienoate, ethyl vanillin, allyl hexanoate and the like. In more preferred embodiment, ethyl vanillin or strawberry flavour are used within the composition. In most preferred embodiment, ethyl vanillin is used as flavouring agent within the concentration range of from about 0.9 mg/ml to about 1.1 mg/ml.

Having described the invention with reference to certain preferred embodiments, other embodiments will become apparent to one skilled in the art from consideration of the specification.

The present invention is further defined by reference to the following examples, which describe in detail methods for the preparation and testing of the said pharmaceutical composition. It will be apparent to those skilled in the art that many modifications, both to materials and methods, may be made without departing from the scope of the invention. The following examples are set out to illustrate the invention and do not limit the scope of the present invention.

TABLE 1
Oral compositions of CBD

| | Content | | |
|---|---|---|---|
| | (one bottle) | 1. conc. | 2. conc. |
| Ingredients | [mg] | [mg/mL] | [mg/mL] |
| CBD | 10000 | 100.00 | 100.00 |
| Neotame | 2.00 | 0.02 | 0.02 |
| Ethyl Vanillin | 100 | 1.00 | 1.00 |
| DL-Alpha Tocopherol | 600 | 6.00 | 6.00 |
| Diethylene glycol mono ethyl | 495 | 4.95 | — |
| ether (Transcutol HP) | | | |
| Lecithin | 50 | — | 0.50 |
| Corn oil | Q.S | Q.S. | Q.S. |

Stability data of the formulations (including Impurity testing) was performed. One-month stability results are as mentioned below:

TABLE 2A
Initial Test results:

| Test | Result |
|---|---|
| Appearance | Light yellow oily liquid. |
| Water content | 0.00% |
| Assay | 96.3% w/w |
| Purity | 99.87% |
| Butyl-CBD content | 0.13% |
| CBD-Hydroquinone Content | 0.00% (above LOD) |
| All other impurities | Not detected (below LOD) |

The stability has been done on the formulation at 40° C./75% relative humidity (RH):

TABLE 2B
Results of Six month stability

| Test | Result | Specification |
|---|---|---|
| Appearance | Light yellow | Light yellow to deep |
| | oily liquid. | yellow liquid |
| Water content | 0.0% | NMT 0.5% |
| Assay | 99.3% | Between 95.0-105.0% |
| | | of theoretical |
| | | concentration |
| | | (100 mg/mL) |
| Purity | 99.85% | NLT 99.0 |
| Butyl-CBD content | 0.13% | — |
| CBD-Hydroquinone Content | 0.02% | — |
| All other impurities | N.D | — |

The stability has been done on the Epidiolex® at 40° C./75% relative humidity (RH).

Initial testing and one-month stability results are as mentioned below:

TABLE 3A
Epidiolex^{R} -Initial analysis:

| Test | Result |
|---|---|
| Appearance | Light yellow oily liquid. |
| Water content | 0.02% |
| Assay | 99.1% |
| Purity | 99.32% |
| Butyl-CBD content | 0.29% |
| CBD-Hydroquinone Content | 0.04% |
| CBDV | 0.35% |

TABLE 3B
Epidiolex^{R} - Six-month test analysis results:

| Test | Result | Specification |
|---|---|---|
| Appearance | Light yellow | Light yellow to deep |
| | oily liquid | yellow liquid |
| Water content | 0.1% | NMT 0.5% |
| Assay | 99.6% | Between 95.0-105.0% |
| | | of theoretical |
| | | concentration |
| | | (100 mg/mL) |
| Purity | 99.24% | — |
| Butyl-CBD content | 0.34% | — |
| CBD-Hydroquinone Content | 0.16% | — |
| CBDV content | 0.36% | — |
| All other impurities | 0.11% | — |

What is claimed is:

1. A stable oral composition comprising of:
a. Cannabidiol
b. DL-α tocopherol
c. a cosolvent selected from Diethylene glycol mono ethyl ether (Transcutol-HPR) or the surfactant agent Lecithin
d. a synthetic sweetener
e. a flavoring agent and
f. Corn oil as a solvent.

2. A stable oral composition comprising of (i) cannabidiol (CBD) at a concentration of from about 95 mg/ml to about 105 mg/ml, (ii) Diethylene glycol mono ethyl ether at a concentration from about 4.5 mg/ml to about 5.5 mg/ml or Lecithin at a concentration from about 0.45 mg/ml to about 0.55 mg/ml, (iii) DL-α tocopherol at a concentration from about 0.54 mg/ml to about 6.6 mg/ml, (iv) synthetic sweetener at a concentration from about 0.018 mg/ml to about 0.022 mg/ml, (v) Flavoring agent at a concentration from about 0.9 mg/ml to about 1.1 mg/ml, and (vi) corn oil q.s. to about 1.0 ml.

3. A stable oral composition of claim 2, wherein the concentration of CBD is about 100 mg/ml±10%.

4. A stable cannabidiol oral composition of claim 2, wherein the concentration of CBD is about 100 mg/ml±10%.

5. A stable oral composition of claim 3, wherein the CBD has a purity of at least about 99.0% of purity and 98.0-105.0% w/w of assay.

6. A stable oral composition of claim 1, wherein the co-solvent is diethylene glycol mono ethyl ether (Transcutol-HP®).

7. A stable oral composition of claim 1, wherein the co-solvent is the surfactant agent lecithin.

8. The stable oral composition of claim 1, wherein the sweetener is selected from the group consisting of sucrose, xylitol, sodium saccharin, neotame, cyclamate, aspartame, acesulfame, saccharin, erythritol and sucralose and combinations thereof.

9. A stable oral composition of claim 1, wherein the flavouring agent is selected from ethyl vanillin, strawberry flavor, and combinations thereof.

10. A stable oral composition of claim 1 wherein: (i) the CBD is at a concentration of between about 95 mg/ml and about 105 mg/ml; (ii) the co-solvent is Diethylene glycol mono ethyl ether (Transcutol-HPR), and present at a concentration of about 4.5 mg/ml to about 5.5 mg/ml; (iii) the sweetener used is preferably neotame at a concentration of about 0.018 mg/ml to about 0.022 mg/ml; (iv) the flavouring agent used is preferably ethyl vanillin at a concentration of about 0.9 mg/ml to about 1.1 mg/ml; (iv) DL-alpha tocopherol at a concentration of about 0.54 mg/ml to about 6.6 mg/ml, and (iv) Com oil, q.s. to about 1.0 ml.

11. A stable oral composition of claim 9, wherein the CBD is at a concentration of about 100 mg/ml.

12. A stable oral composition of claim 9, wherein the CBD has a purity of at least about 98% (w/w).

13. A stable oral composition of claim 9, wherein the CBD is sourced from natural origin.

14. A stable oral composition consisting essentially of:
a. CBD at a concentration of between about 95 mg/ml and about 105 mg/ml, wherein the CBD has a purity of at least about 99.0% of purity and 98.0-105.0% w/w of assay;
b. Diethylene glycol mono ethyl ether (Transcutol-HPR) at a concentration of about 4.95 mg/ml;
c. Neotame at a concentration of about 0.02 mg/ml;
d. DL-alpha tocopherol at a concentration of about 6 mg/ml;
e. Ethyl Vanillin at a concentration of about 1 mg/ml; and
f. Corn oil, q.s. to about 1.0 ml.

15. A stable oral composition consisting essentially of:
a. CBD at a concentration of between about 95 mg/ml and about 105 mg/ml, wherein the CBD has a purity of at least about 99.0% of purity and 98.0-105.0% w/w of assay;
b. Lecithin at a concentration of about 0.50 mg/ml;
c. Neotame at a concentration of about 0.02 mg/ml;
d. D-alpha tocopherol at a concentration of about 6 mg/ml;
e. Ethyl Vanillin at a concentration of about 1 mg/ml; and
f. Corn oil, q.s. to about 1.0 ml.

16. A stable oral composition for use according to claim 14 wherein the oral solution demonstrates stability with less than 0.5% CBDV and other CBD impurities.

17. A method of treatment for a patient in need of a stable oral composition of cannabidiol for use in the treatment of atonic seizures, comprising administering a composition consisting of cannabidiol, corn oil, diethylene glycol mono ethyl ether (Transcutol-HP®) or Lecithin, DL-alpha tocopherol, neotame and ethyl vanillin.
